SEQUENCE LISTING (11 sequences)

SEQ ID NO: 1 - DNA, 21 bp, Artificial Sequence
  Primer
agccccagtgatcttccagat

SEQ ID NO: 2 - DNA, 24 bp, Artificial Sequence
  Primer
aggtggtggaggtgttttacttct

SEQ ID NO: 3 - DNA, 23 bp, Artificial Sequence
  Probe
ccctggacaaaccatgccaccaa

SEQ ID NO: 4 - DNA, 37 bp, Artificial Sequence
  Probe
gtggagtatttgatagtgtattaaccttatgtgtgac

SEQ ID NO: 5 - DNA, 37 bp, Artificial Sequence
  Probe
ttccagcagtgtcacagcaccctagaaccaaatccag

SEQ ID NO: 6 - DNA, 37 bp, Artificial Sequence
  Probe
cagatagccctggacaaacaatgccacgaagcagaag

SEQ ID NO: 7 - DNA, 37 bp, Artificial Sequence
  Probe
tactcccctgccctcaacaagatgttttgccaactgg

SEQ ID NO: 8 - DNA, 35 bp, Artificial Sequence
  Probe
atttcttccatactactacccatcgacctctcatc

SEQ ID NO: 9 - DNA, 37 bp, Artificial Sequence
  Probe
atgaggccagtgcgccttggggagacctgtggcaagc

SEQ ID NO: 10 - DNA, 37 bp, Artificial Sequence
  Probe
gaaaggacaagggtggttgggagtagatggagcctgg

SEQ ID NO: 11 - DNA, 314 bp, Homo Sapiens
  misc_feature (246)..(253): n corresponds to a nucleotide of unknown identity.
ccagtggtatagaaatcttcgatttttaaattcttaattttaggttgcagtttcatcactgtctgcggtaatcaagtttttagaactcttatcagatgattccaactttggacagtttgaactgactacttttgacttcagccagtatatgaaattggatattgcagcagtcagagcccttaacctttttcaggtaaaaaaaaaaaaaaaaaaaaaaaaaagggttaaaaatgttgattggttaannnnnnnngacagatagtgaagaaggcttagaaaggagctaaaagagttcgacatcaatattagacaag

(19) United States
(12) Patent Application Publication
(10) Pub. No.: US 2004/0259101 A1
(43) Pub. Date: Dec. 23, 2004

(54) Methods for disease screening

(76) Inventor: Anthony P. Shuber, Mendon, MA (US)

Correspondence Address: TESTA, HURWITZ & THIBEAULT, LLP, HIGH STREET TOWER, 125 HIGH STREET, BOSTON, MA, 02110, US

(21) Appl. No.: 10/601,132

(22) Filed: Jun. 20, 2003

(51) Int. Cl. C12Q 1/68

(52) U.S. Cl. 435/6

(57) ABSTRACT

Methods of the invention generally include measuring a quantitative amount of nucleic acid in a patient sample, and optionally, performing additional disease testing on the patient. Methods of the invention are useful in screening disease in a patient, such as cancer.

### FIELD OF THE INVENTION

This invention generally relates to methods for screening for disease in a patient. More specifically, this invention relates to the quantitative analysis of nucleic acids in a patient sample, and optionally, performing additional disease testing.

### BACKGROUND OF THE INVENTION

Current methods of disease screening involve examining or testing individuals for early stages of disease. Preferably, individuals are screened for disease even before they exhibit symptoms. Early-stage screening is important because early diagnosis of a disease can make treatment easier and more effective, and can decrease mortality. Additionally, early treatment of a disease may help to slow, stop, or even reverse disease progression so that an individual never becomes symptomatic.

Current disease screening methods include, for example, invasive tests and cellular-based assays. Examples of invasive tests include physical examination and biopsy of potentially-cancerous tissue. Examples of cellular-based assays include analysis of DNA, RNA, chromosomes, proteins, and certain metabolites to detect heritable disease-related genotypes, mutations, phenotypes, or karyotypes for clinical purposes. Genetic assays for cancer often involve probing specific genes for previously identified mutations. For example, a number of genetic mutations, including alterations in the BAT-26 segment of the MSH2 mismatch repair gene, the p53 gene, the ras oncogene, and the APC tumor suppressor gene have been associated with the multi-step pathway leading to cancer.

Known screening methods contain a number of practical limitations. For example, invasive cancer screening procedures are often expensive and can result in significant patient discomfort or possibly severe medical complications. Further, the cost of commercially available genetic assays for disease screening can range from hundreds to thousands of dollars, depending on the sizes of the genes and the numbers of mutations tested. Accordingly, there is a need in the art for relatively simple and inexpensive screening methods that can be administered to a patient prior to performing additional disease testing. Such methods are provided herein.

### SUMMARY OF THE INVENTION

The present invention is based on the observation that the amount of nucleic acid in a patient sample is indicative of the presence of disease in the patient. Accordingly, methods of the invention comprise quantifying an amount of nucleic acid in a patient sample. If the amount of nucleic acid in the sample is greater than a predetermined threshold amount, then the patient is identified as a candidate for additional disease testing. The predetermined threshold amount is preferably set so that patient samples having an amount of nucleic acid lower than the predetermined threshold amount can be identified as being relatively disease-free. Methods of the invention are useful as screening techniques for any disease, including cancer, such as, but not limited to, colorectal cancer.

Methods of the invention can be used to identify a subset of a patients in a population that are relatively disease-free. In certain embodiments, this patient subset does not undergo additional disease testing, although additional disease testing may be performed if desired. In one embodiment, the predetermined threshold amount is set so that approximately 10-20% of patients in a population can be identified as being relatively disease-free using methods of the invention. Therefore, the present invention provides cost-effective screening methods to determine if a patient is a candidate for additional disease testing.

Methods of the invention provide that the quantitative amount of nucleic acid in a sample is indicative of disease status of the patient from whom the sample was obtained. According to the invention, tissue or body fluid samples, especially those described below, contain shed cellular debris. In diseases such as cancer in which cells undergo uncontrolled cell growth and the cell cycle mechanisms are destroyed or impaired, it is believed (without any intention of being bound by the theory) that samples containing cellular debris from such patients have an increased amount of nucleic acid relative to samples from certain healthy patients. As a result, it has been discovered that patients can be screened for disease by quantitatively measuring an amount of nucleic acid in a patient sample.

Methods of the invention are practiced by quantifying an amount of nucleic acid in a sample and comparing the measured amount to a predetermined threshold amount. A positive screen represents a measured amount of nucleic acid greater than the predetermined threshold amount. A negative screen represents a measured amount of nucleic acid lower than the predetermined threshold amount.

The predetermined threshold amount can be determined by empirical means. For example, the predetermined threshold amount can be determined by amplifying a particular genetic locus in a sample from each of a population of normal and diseased patients and quantitatively analyzing the amount of DNA in each of the patients in the population. The predetermined threshold amount is preferably set below the measured amount of nucleic acid in any of the diseased patient samples. Once the predetermined threshold amount has been determined, it can be used as the basis for further screening.

There are numerous ways in which the amount of nucleic acid in a patient sample is quantitatively measured. In certain embodiments, the amount of nucleic acid in a patient sample is quantitatively measured by calculating a number of genome equivalents (as used herein, genome equivalents is abbreviated “GE”) in a sample. For example, one GE is equivalent to the amount of genomic DNA present in one normal cell. Thus, as one non-limiting example, a measurement of 100 GEs in a sample indicates that the sample contains approximately the same amount of DNA as would be found in 100 cells. In certain circumstances, the number of GEs can be related to the number of copies of a particular segment of the genome, such as a particular gene, exon, or intron. The number of GEs can be calculated by amplifying one or more genetic loci thought to be present in a sample and quantitatively analyzing the amount of genomic DNA in the sample through any quantitative process known in the art. In certain embodiments of the invention, one GE is the equivalent of about 7 picograms of DNA. In some embodiments, an amplification reaction is conducted at a single genomic locus to amplify a fragment of a specific length. Typically, fragments of 200 bp or less at the same genomic locus are amplified. There is generally a one-to-one correspondence between the amplification of a single 200 bp fragment and one genome equivalent. Therefore, quantitative PCR will determine how many 200 bp fragments of a specific site were available originally in the sample, and thus, the number of GEs in the sample. GE scores will vary depending on a number of factors, including, but not limited to, preparation methods, amplification methods, and quantitative analysis methods. In certain embodiments, the quantity of human genomic DNA (or other patient DNA, such as animal DNA) in a heterogeneous sample comprising shed cells or cellular debris is measured.

Additional disease testing of the invention includes, but is not limited to, genetic assays, diagnostic evaluation, and physical examination. Methods of the invention are useful as general disease screening techniques, and are useful as screens for a wide-range of disease states. Methods of the inventions are also useful as screening techniques for the presence of cancer and pre-cancer, and are especially useful as screening techniques for colorectal cancer and pre-cancer.

In one aspect of the invention, the invention provides methods for screening a patient for the presence of disease including the steps of measuring a quantitative amount of nucleic acid in a patient sample comprising shed cells or cellular debris, and identifying the patient as a candidate for additional disease testing if the amount of nucleic acid is above a predetermined threshold amount.

This aspect of the invention can have any of the following features. The nucleic acid can be genomic DNA. The measuring step can include determining a number of genome equivalents. The method can further include the step of performing an assay on a sample from the patient if the patient is identified as a candidate for additional disease testing. This assay can be a DNA integrity assay, mutation detection assay, enumerated loss of heterozygosity (LOH) assay, expression assay, and/or fluorescent in-situ hybridization (FISH) assay. The assay can detect mutations at any genetic locus such as, but not limited to, p53, ras, APC, DCC, and/or BAT-26. The method can further include the step of performing a diagnostic examination on the patient if the patient is identified as a candidate for additional disease testing. The step of performing a diagnostic examination can be a colonoscopy, a sigmoidoscopy, a fecal occult blood testing and/or an upper gastrointestinal evaluation. The patient sample can be stool, sputum, pancreatic fluid, bile, lymph, blood (such as blood serum or blood plasma), urine, cerebrospinal fluid, seminal fluid, saliva, breast nipple aspirate, and/or pus. The disease can be cancer or pre-cancer. The cancer can be colorectal cancer, lung cancer, esophageal cancer, prostrate cancer, stomach cancer, pancreatic cancer, liver cancer, and/or lymphoma.

In another aspect of the invention, the invention provides methods for screening a patient for the presence of abnormal proliferating cells including the steps of measuring a quantitative amount of nucleic acid in a patient sample including shed cells or cellular debris, and identifying a positive screen as a sample in which the amount of nucleic acid is above a predetermined threshold amount.

This aspect of the invention can include any of the features described above or below. The nucleic acid can be genomic DNA. The measuring can be determining a number of genomic equivalents. The method can further include the step of performing an assay on a sample from the patient if a positive screen is identified in the identifying step. This assay can be a DNA integrity assay, mutation detection, enumerated LOH, expression assays, and FISH. This assay also can be one that detects mutations at a genetic locus including p53, ras, APC, DCC, and/or BAT-26. The method can further include the step of performing a diagnostic examination on the patient if a positive screen is identified in the identifying step. The step of performing a diagnostic examination can be a colonoscopy, a sigmoidoscopy, a fecal occult blood testing and/or an upper gastrointestinal evaluation. The patient sample can be stool, sputum, pancreatic fluid, bile, lymph, blood (such as blood serum or blood plasma), urine, cerebrospinal fluid, seminal fluid, saliva, breast nipple aspirate, and/or pus.

In a further aspect of the invention, the invention provides methods for diagnosing the state of health of a patient including the steps of measuring a quantitative amount of nucleic acid in a patient sample including shed cells or cellular debris, and performing an assay on a sample from the patient if the amount of nucleic acid is above a predetermined threshold amount such that the state of health of a patient is determined.

This aspect of the invention can have any of the following or preceding features. The nucleic acid can be genomic DNA. The measuring can include determining a number of genome equivalents. The assay can be a DNA integrity assay, mutation detection, enumerated LOH, expression assays, and/or FISH. The assay can detect mutations at a genetic locus including p53, ras, APC, DCC, and/or BAT-26. The method can further include performing a diagnostic examination on the patient. The diagnostic examination can be a colonoscopy, a sigmoidoscopy, a fecal occult blood testing, and/or an upper gastrointestinal evaluation. The patient sample can be stool, sputum, pancreatic fluid, bile, lymph, blood (such as blood serum or blood plasma), urine, cerebrospinal fluid, seminal fluid, saliva, breast nipple aspirate, and/or pus.

The present invention is pointed out with particularity in the appended claims. The objects and advantages of the invention described above, as well as further objects and advantages of the invention, are better understood by reference to the following detailed description taken in conjunction with the accompanying drawings.

### DESCRIPTION OF THE DRAWINGS

FIG. 1 is a flowchart representation of method steps for diagnosing disease in a patient in accordance with one embodiment of the invention.

FIG. 2 is a flowchart representation of method steps for diagnosing disease in a patient in accordance with another embodiment of the invention.

FIG. 3 is a flowchart representation of method steps for diagnosing disease in a patient in accordance with a further embodiment of the invention.

FIG. 4 is a flowchart representation of method steps for diagnosing disease in a patient in accordance with another embodiment of the invention.

FIG. 5 is a flowchart representation of method steps for diagnosing colorectal cancer in accordance with one particular embodiment of the invention.

### DETAILED DESCRIPTION OF THE INVENTION

Methods of the present invention are useful in screening for disease in a patient. Methods of the invention provide that the amount of nucleic acid in a sample comprising shed cellular material is indicative of the disease status of the patient from whom the sample was obtained. In general, methods of the invention comprise measuring the quantitative amount of nucleic acid in a sample from a patient. If the measured amount of nucleic acid in the sample is greater than a predetermined threshold amount, then additional disease testing is performed on the patient. The additional disease testing includes, for example, genetic assays and physical examination. If the measured amount of nucleic acid in the sample is lower than a predetermined threshold amount, then no additional disease testing is necessary, although it can be conducted if desired. Accordingly, the present invention provides relatively cost-effective screening methods that can be administered to a patient prior to performing additional disease testing on the patient.

As discussed herein, the present invention is based on the observation that the amount of nucleic acids in a patient sample is indicative of the presence of disease. In one embodiment, methods of the invention comprise screening a patient sample by conducting an amplification reaction using as a template a nucleic acid suspected or expected to be in the sample; measuring a quantitative amount of amplification product obtained; comparing the amount of amplification product obtained to a predetermined threshold amount; and identifying the patient as a candidate for additional disease testing if the amount of amplification product is greater than the predetermined threshold amount. In certain embodiments, a predetermined threshold amount is determined by empirical means. Also in certain embodiments, a predetermined threshold amount is determined by reference to the art.

One method of the invention includes conducting in a tissue or body fluid sample an amplification reaction using as a template a nucleic acid locus suspected to be in the sample. If the amount of amplification product (amplicon) is greater than a predetermined threshold amount, then additional testing may be performed on a patient. In the case of DNA, the amplification reaction can be a polymerase chain reaction (PCR). Methods for conducting PCR are provided in U.S. Pat. No. 4,683,202, incorporated by reference herein. In the case of RNA, the amplification reaction can be reverse transcriptase PCR. Primers are designed to amplify the locus or loci chosen for analysis. For purposes of the invention a “genomic locus” is any genetic element, including, but not limited to, a coding region of a gene, a non-coding nucleic acid region, a regulatory element of a gene, an intron, or RNA. It is not required that the target genomic loci be associated with any specific disease, as an increase in amplifiable nucleic acid is itself diagnostic.

Any tissue or body fluid specimen may be used as a patient sample according to methods of the invention. Samples typically include those generally free of intact, healthy cells, which include, but are not limited to, luminal fluid, blood (such as blood plasma or blood serum), urine, bile, pancreatic juice, stool, sputum, pus, and the like. Methods of the invention can be practiced using patient samples that are most likely to contain sloughed cellular debris. Such samples include, but are not limited to, stool, blood serum or plasma, sputum, pus, pancreatic fluid, bile, saliva, lymph, urine, cerebrospinal fluid, seminal fluid, and breast nipple aspirate. Methods of the invention are especially useful to detect disease in biological samples comprising shed cells or cellular debris. For example, the presence in a patient stool sample of high amounts of nucleic acid, such as DNA, above a predetermined threshold is indicative that the patient has a disease, and is identified for further testing. Some embodiments of the invention for use on a stool sample include obtaining a representative stool sample. Exemplary methods for preparing a stool sample are disclosed in U.S. Pat. Nos. 5,741,650 and 5,952,178, each of which is incorporated by reference herein.

Methods of the invention are practiced by measuring the quantitative amount of nucleic acids in a patient sample. In certain embodiments, the nucleic acid being quantitatively measured by methods of the invention is DNA. However, nucleic acids measured by the invention are not limited to any particular type of nucleic acid and include, for example, but are not limited to, total genome DNA, cDNA, RNA, mRNA, tRNA, and rRNA. In a particular embodiment, the nucleic acid being analyzed is selected from a coding region of a gene, or portion thereof, a noncoding nucleic acid region, or portion thereof, a regulatory element of a gene or a portion thereof, and an unidentified fragment of genomic DNA. Also in certain embodiments, the nucleic acid being interrogated is RNA. As is appreciated by the skilled artisan, any genomic locus is amenable to screening according to the invention. The particular locus or loci chosen for analysis depends, in part, on the disease being screened, and the convenience of the investigator. It is not necessary that the locus or loci chosen for analysis be correlated with any specific disease because methods of the invention contemplate measuring the amount of nucleic acid in a sample as an indicator of overall disease status. However, disease-associated loci (those in which a mutation is indicative, causative, or otherwise evidences a disease) can be used. Examples of disease-associated loci include p53, apc, MSH-2, dcc, scr, c-myc, B-catnenin, mlh-1, pms-1, pms-2, pol-delta, and bax.

The quantitative amount of amplification product may be determined by any suitable or convenient means. Preferably, the amount of amplification product is determined by quantitative PCR, for example, by using real-time PCR machines, such as Biorad Corporation's iCycler iQ Real Time PCR Detection System, but any quantitative system or means may be used. The amplification reaction itself can be any means for amplifying nucleic acid, including, but not limited to PCR, RT-PCR, OLA, rolling circle, single base extension, and others known in the art. Methods of the invention are useful with any platform for the identification, amplification, sequencing, or other manipulation of nucleic acids.

In certain embodiments, methods of the invention comprise determining an amount of amplifiable nucleic acid in a biological sample, and determining whether that amount is consistent with an amount expected in a normal sample. In many biological samples, especially heterogeneous samples, there may be no detectable amplification product. That is especially true when longer fragments are used as templates for amplification. Generally, the probability that any given set of PCR primers will amplify a DNA fragment having a length exceeding the primer distance is expressed as

% of Fragments Amplified=(FL−PD)/(FL+PD)

wherein FL is fragment length (in base pairs) and PD is primer distance (in base pairs). This equation assumes that sample DNA fragment lengths are uniformly distributed (i.e., there is no favored locus at which breaks occur). The lengths of fragments to be amplified in this assay may be varied, but are preferably less than 200 bp in length.

Methods of the invention can be carried out by hybrid capture. For example, hybrid capture and subsequent analysis of the captured fragments can be used to determine the quantitative amount of nucleic acid in a patient sample. In certain embodiments, a hybrid capture probe is used to anchor a target sequence, preferably on a solid support (e.g., beads). Capture probes can be pairs of forward and reverse primers, or they can be signal amplification probes, such as those used in Ligase Chain Reaction (LCR), and others used in the identification of sequences. The probes hybridize along the target fragment. Thus, by analyzing samples for the presence of the probes, one can determine the quantitative amount of nucleic acid present in the sample. This can be done in numerous ways, including, but not limited to, hybrid capture, PCR, LCR, strand displacement, branched chain, or other assays known in the art that incorporate hybrid probes or primers to quantitate a sequence. A sample containing a quantitative amount of nucleic acid above a predetermined threshold amount represents a positive screen according to the invention.

There are numerous ways in which the quantitative amount of nucleic acid in a sample is calculated. In certain embodiments, the amount of nucleic acid in a patient sample is accomplished by measuring a number of GEs in a sample. One GE is equivalent to the amount of genomic DNA present in one normal cell. For example, a measurement of 100 GEs in a sample indicates that the sample contains approximately the same amount of DNA as would be found in 100 cells. In certain circumstances, the number of GEs can be related to the number of copies of a particular segment of the genome, such as a particular gene, exon, or intron. The number of GEs can be calculated by amplifying one or more genetic loci thought to be present in a sample and quantitatively analyzing the amount of genomic DNA in the sample through any quantitative process known in the art (for example, by comparing this amount to a known standard amount of DNA). In certain embodiments of the invention, one GE is the equivalent of about 7 picograms of DNA. In some embodiments, an amplification reaction is conducted at a single genomic locus to amplify a fragment of a specific length. Typically, fragments of 200 bp or less at the same genomic locus are amplified. There is generally a one-to-one correspondence between the amplification of a single 200 bp fragment to one genome equivalent. Therefore, quantitative PCR will determine how many 200 bp fragments of a specific site were available originally in the sample, and thus, the number of GEs in the sample. GE scores will vary depending on a number of factors, including, but not limited to, preparation methods, amplification methods, and quantitative analysis methods, and also on the desired likelihood of false-negative results.

A positive screen of the invention results when the measured amount of nucleic acid in a patient sample is greater than a predetermined or threshold amount. In one embodiment, the predetermined threshold amount can be determined by amplifying a particular genetic locus in a population of normal and diseased patients. The predetermined threshold amount can be determined empirically by determining the amount of nucleic acid in a sample from each of the patients in a population and setting the predetermined threshold amount based on the amount of nucleic acid in any of the patient samples (for example, below the lowest amount of nucleic acid detected in any diseased patient). Once determined, this threshold can be used as the basis for additional disease testing. In one particular embodiment of the invention, the threshold amount is approximately 500 GEs, although as discussed above, these scores will vary depending on a number of factors. By way of further examples, and without any intent of limiting the scope of the invention to such examples, the threshold amount can be any number of GEs in the ranges of, for example, 10 to 10,000 GEs, 100 to 10,000 GEs, 200 to 8,000 GEs, 1,000 to 3,000 GEs, 1,000 to 100,000 GEs, and/or any integer between 10 and 10,000, or any range between any of two integers from 10 to 10,000.

In accordance with the invention, if the amount of nucleic acid in the patient sample is greater than the predetermined threshold amount, then the patient is identified as a candidate for additional disease testing. If the amount of nucleic acid in the sample is lower than the predetermined or threshold amount, then no additional disease testing is necessary, although such testing may be performed if desired. As such, the present invention provides relatively cost-effective screening methods that can be administered to a patient prior to performing additional disease testing. It should be understood that there are several ways to set a predetermined threshold amount. For example, one can set the threshold below the amount of nucleic acid (e.g., GEs) in a sample from the diseased patient exhibiting the lowest amount of nucleic acid in a group of patients (both normal and diseased) or below the amount of nucleic acid in a sample from the normal patient exhibiting the highest amount of genomic DNA that is not greater than the amount of nucleic acid of the diseased patient exhibiting the lowest amount of nucleic acid in a group of patients. Moreover, the predetermined threshold amount can be set below either of these two numbers to reduce the likelihood of false-negative results or can be set above either of these two numbers to reduce the likelihood of false-positive results.

Methods of the invention are useful as screening methods. Accordingly, such methods are used to screen or to “qualify” patient samples for further analysis (e.g., genetic, biochemical, cytological, or other analyses). Often it is desirable to perform follow-up testing on a patient in order to confirm a suspected disease state. Such follow-up procedures are determined based upon the disease state being interrogated.

Additional disease testing of the invention includes, but is not limited to, screening assays, diagnostic evaluation, and physical examination. Additional testing of the invention includes mutation assays to detect a cancer marker (e.g., a DNA mutation) in a sample from a patient. Such mutation assays include, but are not limited to, assays for the detection of mutations at the p53 tumor suppressor locus, in ras genes, in APC and DCC tumor suppressor genes, and in the BAT-26 segment of the MSH2 mismatch repair gene. For purposes of the present invention, a mutation is a deletion, addition, substitution, rearrangement, or translocation in a nucleic acid. Numerous mutational analyses are known in the art and include, for example, U.S. Pat. No. 5,670,325, incorporated by reference herein.

Additional disease testing of the invention preferably comprises DNA integrity assays, as described in co-owned, co-pending U.S. patent application Ser. No. 09/455,950, incorporated by reference herein. It has been recognized that DNA obtained from exfoliated normal (non-cancerous) cells is different than DNA obtained from exfoliated cancer or precancer cells. Normal exfoliated cells typically have undergone apoptosis, and thus produce cells or cellular debris (depending upon the stage of apoptosis) comprising DNA that has been substantially degraded. Exfoliated cancer or precancer cells typically have not undergone apoptosis, and such cells or their debris, while producing some very small fragments as a result of degradation in the sample, typically also contain a higher proportion of large DNA fragments (compared to those observed in cells or debris from exfoliated normal cells). The difference in DNA integrity between normal and abnormal cells is a marker for the presence of cancer or precancer in a sample comprising exfoliated cells.

In one embodiment, the additional disease testing component of the invention preferably comprises detecting in a biological sample one or more DNA fragment(s) of a length that would not be substantially present in noncancerous cells or cellular debris. There is no upper limit on these fragments, as all that is necessary is that the fragment be larger than an apoptotic fragment (i.e., about 200 bp). Typically, however, fragments indicative of cancer or precancer cells are between about 200 and about 3500 base pairs, and ideally between about 500 and about 2500 base pairs, such as, for example, a 1000 or 1300 base pair fragment. In certain embodiments, gel electrophoresis, affinity chromatography, or mass spectrometry are used to detect large DNA fragments (fragments comprising greater than about 200 base pairs). In one embodiment, the presence of large DNA fragments in a stool sample is indicative of colorectal cancer in a patient.

In certain embodiments, the additional disease testing component of the invention comprises amplifying nucleic acids in a representative stool sample using human-specific primers, and detecting amplicons having greater than about 200 base pairs, and preferably about 500 or more base pairs. In certain embodiments, amplification is accomplished by PCR using forward and reverse primers directed against human-specific nucleic acid-fragments, and spaced apart to provide a lower limit on the resulting amplicons. The presence of amplicons greater than about 200 base pairs in length is indicative of template nucleic acid in the sample of that length (or longer). According to the additional disease testing component of the invention, such long sequences represent a positive screen and are indicative of cancer or precancer.

Additional testing of the invention also includes, for example, but is not limited to, performing an expression assay, a FISH assay, or an assay for enumerated LOH. Enumerated LOH assays are described in, for example, U.S. Pat. Nos. 6,203,993 and 6,300,077, each of which are incorporated by reference herein. Additional testing of the invention further includes detection of extracellular indicia of disease. Such detection methods include, for example, but are not limited to, fecal occult blood testing (FOBT) and detection of elevated antigen levels, such as carcinoembryonic or prostate-specific antigen.

Additional testing of the invention also includes performing a diagnostic examination on a patient. Examples of diagnostic examination include, but are not limited to, colonscopy, virtual colonscopy, sigmoidoscopy, flexible sigmoidoscopy, upper gastrointestinal evaluation, digital rectal examination, mammography, breast self-examination, computed tomography (CT) imaging, magnetic resonance imaging (MRI), positron emission tomography (PET), x-ray, ultrasound, biopsy, surgery, endoscopy, laparoscopy, and endoscopic retrograde cholangiopancreatography (ERCP).

In one embodiment, additional disease testing or follow-up analysis is used to determine where the disease resides. However, the general disease screen is effective independent of the location of the disease and the specimen taken for analysis. Thus, for example, while measurement of nucleic acid in stool is predictive of disease generally, it does not necessarily indicate that the disease is of gastrointestinal origin. However, follow-up testing or additional disease testing are used to identify the disease.

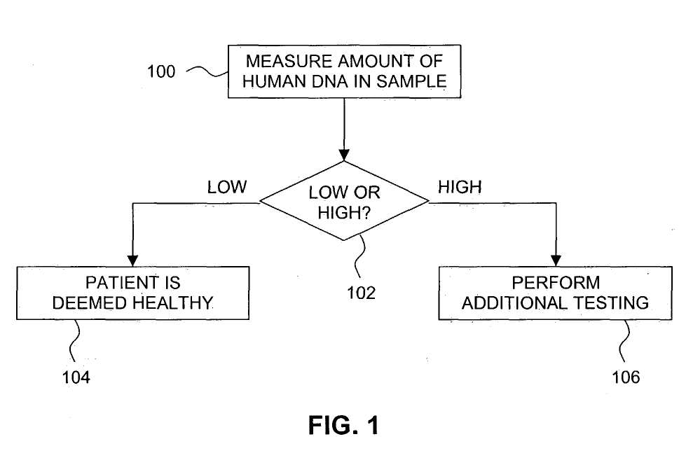

Methods of the invention may be practiced in accordance with protocols for diagnosing disease in a patient. In one embodiment, the quantitative amount of nucleic acid in a sample is measured as part of a protocol for diagnosing disease in a patient. FIGS. 1-5 describe particular examples of protocols for diagnosing disease in a patient. Although protocols in accordance with the invention are described with reference to specific embodiments, it should be understood by those skilled in the art that various changes in form and detail may be made therein without departing from the spirit and scope of the invention.

The flowchart in FIG. 1 describes a basic implementation of the present invention for diagnosing disease in a patient. At step 100, the quantitative amount of human DNA present in a the patient sample is determined. In certain embodiments, the amount of human DNA is determined as a number of GEs in the sample. At step 102, if the amount of human DNA is lower than a predetermined threshold amount, then the patient is deemed healthy (or disease-free) in step 104. Also at step 102, if the amount of human DNA is higher than a predetermined threshold amount, then additional disease testing is performed on the patient in step 104. This additional disease testing can be any of those described herein or any other disease testing known in the art.

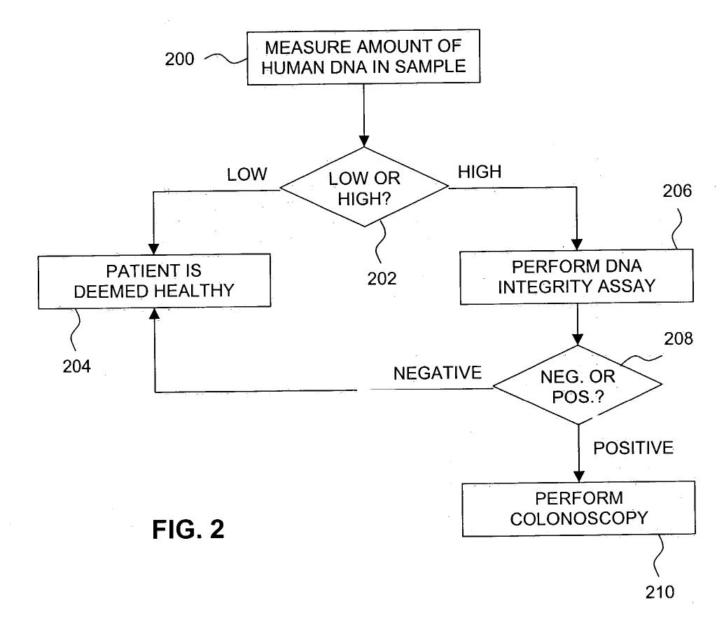

The flowchart in FIG. 2 describes a more detailed implementation of the present invention for diagnosing disease in a patient. At step 200, the amount of human DNA in a sample is determined. In certain embodiments, the amount of human DNA is determined as a number of GEs in the sample. At step 202, if the amount of human DNA is lower than a predetermined threshold amount, then the patient is deemed healthy (or disease-free) in step 204. Also at step 102, if the amount of human DNA is higher than a predetermined threshold amount, then a DNA integrity test is performed on a sample from the patient in step 206. At step 208, if a positive screen is obtained from the DNA integrity test, then a colonoscopy is performed on the patient in step 210. Also at step 208, if a negative screen is obtained from the DNA integrity test, then the patient is deemed healthy (or disease-free) in step 204.

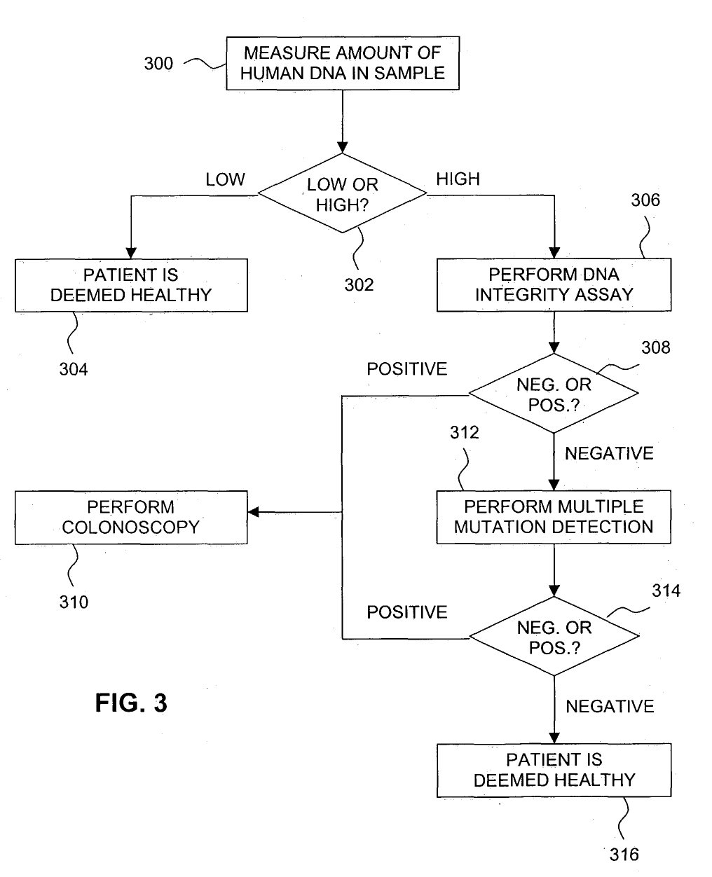

The flowchart in FIG. 3 describes a further detailed implementation of the present invention for diagnosing disease in a patient. At step 300, the amount of human DNA in a sample is determined. In certain embodiments, the amount of human DNA is determined as a number of GEs in the sample. At step 302, if the amount of human DNA is lower than a predetermined threshold amount, then the patient is deemed healthy (or disease-free) in step 304. Also at step 302, if the amount of human DNA is higher than a predetermined threshold amount, then a DNA integrity test is performed on a sample from the patient in step 306. At step 308, if a positive result is obtained from the DNA integrity test, then a colonoscopy is performed on the patient in step 310. Also at step 308, if a negative result is obtained from the DNA integrity test, then a mutation detection assay is performed on a sample from the patient in step 312. At step 314, if a positive result is obtained from the mutation detection assay (i.e. a mutation is detected), then a colonoscopy is performed on the patient in step 310. Also at step 314, if a negative result is obtained from the mutation detection assay (i.e., a mutation is not detected), then the patient is deemed healthy (or disease-free) in step 316.

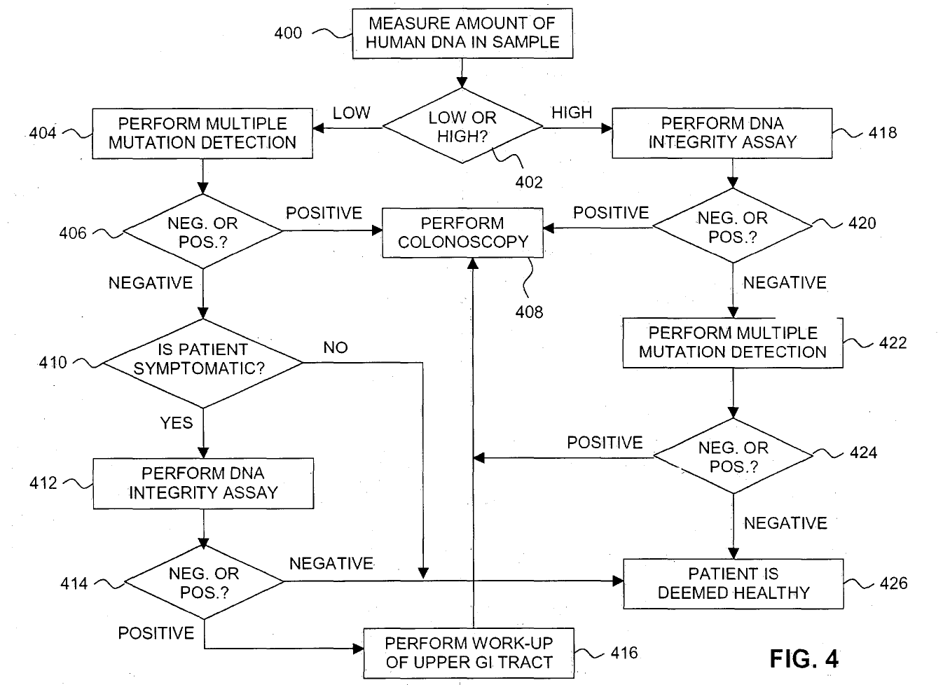

The flowchart in FIG. 4 describes an even further detailed implementation of the present invention for diagnosing disease in a patient. At step 400, the amount of human DNA in a sample is determined. In certain embodiments, the amount of human DNA is determined as a number of GEs in the sample. At step 402, if the amount of human DNA is lower than a predetermined threshold amount, then a mutation detection assay is performed on the patient in step 404. At step 406, if a positive result is obtained from the mutation detection assay, then a colonoscopy is performed on the patient in step 408. Also at step 406, if a negative result is obtained from the mutation detection assay, then the patient is examined for symptoms of disease in step 410. If the patient is not symptomatic for disease, then the patient is deemed healthy (or disease-free) in step 426. If the patient is symptomatic for disease, then a DNA integrity assay is performed on a sample from the patient in step 412. At step 414, if a positive result is obtained from the DNA integrity test, then an upper gastrointestinal work-up is performed on the patient in step 416, followed by a colonoscopy in step 408. Also at step 414, if a negative result is obtained from the DNA integrity test, then the patient is deemed healthy (or disease-free) in step 426.

At step 402, if the amount of human DNA is higher than a predetermined threshold amount, then a DNA integrity test is performed on a sample from the patient in step 418. At step 420, if a positive result is obtained from the DNA integrity test, then a colonoscopy is performed on the patient in step 408. Also at step 420, if a negative result is obtained from the DNA integrity test, then a mutation detection assay is performed on a sample from the patient in step 422. At step 424, if a positive result is obtained from the mutation detection assay, then a colonoscopy is performed on the patient in step 408. Also at step 424, if a negative result is obtained from the mutation detection assay, then the patient is deemed healthy (or disease-free) in step 426.

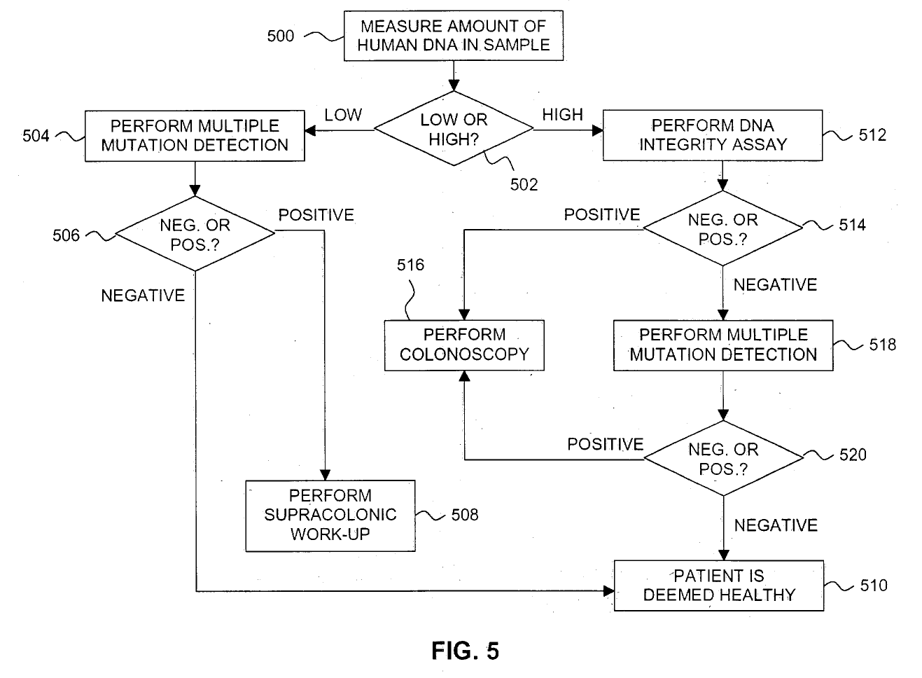

The flowchart in FIG. 5 describes a detailed implementation of the present invention for diagnosing colorectal cancer in a patient. At step 500, the amount of human DNA in a sample is determined. In certain embodiments, the amount of human DNA is determined as a number of GEs in the sample. At step 502, if the amount of human DNA is lower than a predetermined threshold amount, then a mutation detection assay is performed on the patient in step 504. At step 506, if a positive result is obtained from the mutation detection assay, then a supracolonic work-up is performed on the patient in step 508. At step 506, if a negative result is obtained from the mutation detection assay, then the patient is deemed healthy in step 510.

At step 502, if the amount of human DNA is higher than a predetermined threshold amount, then a DNA integrity test is performed on a sample from the patient in step 512. At step 514, if a positive result is obtained from the DNA integrity test, then a colonoscopy is performed on the patient in step 516. Also at step 514, if a negative result is obtained from the DNA integrity test, then a mutation detection assay is performed on a sample from the patient in step 518. At step 520, if a positive result is obtained from the mutation detection assay, then a colonoscopy is performed on the patient in step 516. Also at step 520, if a negative result is obtained from the mutation detection assay, then the patient is deemed healthy in step 510.

The present invention provides relatively cost-effective screening methods that can be administered to a patient prior to performing additional disease testing. Methods of the invention are useful as general disease screening techniques, and are useful as screens for a wide-range of disease states. Methods of the inventions are also useful as screening techniques for the presence of cancer and pre-cancer, and are especially useful as screening techniques colorectal cancer and pre-cancer. In addition to colorectal cancers, methods of the invention are useful to screen for other cancers, for example, as screening techniques for lymphomas, stomach cancers, lung cancers, liver cancers, pancreas cancers, prostrate cancers, kidney cancers, testicular cancers bladder cancers, gallbladder cancers, uterine cancers, and ovarian cancers. Methods of the invention are also useful for screening for the presence of cancerous or precancerous lesions in a patient, including adenomas.

In addition to cancer, methods of the invention are useful, for example, as screening techniques for diseases such as inflammatory bowel syndrome, inflammatory bowel disease, Crohn's disease, respiratory distress syndrome, and others in which the performance of diagnostic procedures followed by the performance of screening methods of the invention would be effective in the detection of disease. Furthermore, the methods of the invention are also useful for detecting an indicator of the presence of an infectious agent, including, but not limited to, a virus, bacterium, parasite, or other microorganism. The invention is equally applicable to human and to veterinary uses. Accordingly, “patient” as discussed herein is intended to included humans and other animals.

In one embodiment, methods of the invention are used to monitor the progress of a disease in a patient or in populations of patients. Such longitudinal monitoring provides information on the degree to which the quantitative amount of nucleic acid in samples is increasing or decreasing as disease progresses or recedes. Longitudinal monitoring of the total genomic DNA in a patient sample can be done without reference to an external predetermined threshold and, instead, uses amounts determined at prior time point(s) as the predetermined threshold. For example, the nucleic acid in a patient's sample can be quantified at two or more time points. If the amount of nucleic acid increases from one or more previous time points, the patient can be tested with follow up additional disease testing. Alternatively, the amount of nucleic acid in a patient's sample can be quantified at two or more time points, and, if the amount of nucleic acid decreases from one or more previous time points and if the patient is being treated for a disease, the patient may show signs of partial or total abatement, alleviation, or treatment of a disease. Generally, such longitudinal monitoring can be used to assess the efficacy of treatments (e.g., chemotherapy, antibiotics), and the response of patients to therapeutic interventions. Methods of the invention can also be used to predict disease flare-up. For example, monitoring fluctuations in the quantitative amount of nucleic acids in samples from diseased patients, such as patients with inflammatory bowel disease, is useful to predict onset of disease episodes. According to the invention, episodic occurrence of symptoms is tied to an increase in the quantitative amount of nucleic acids in patient samples.

Methods of the invention are also useful to establish patient databases. Such databases are used to identify specific patients, to establish where a particular patient fits in a disease continuum, to follow trends in disease, to predict disease onset, or to compile statistics on disease frequency, to monitor patient progress and treatment efficacy, and the like.

Methods of the invention are also useful to predict risk for disease and to predict disease onset. Levels of nucleic acids in patient samples are useful as a quantitative or quasi-quantitative measure of disease. Thus, the level of, for example, nucleic acids obtained from a patient sample is compared to predetermined threshold amounts representing various stages of disease in order to assess the patient's disease state and prognosis.

The following examples provide further details of methods according to the invention. For purposes of exemplification, the following examples provide details of the use of methods of the present invention in colorectal cancer detection. Accordingly, while exemplified in the following manner, the invention is not so limited and the skilled artisan will appreciate its wide range of application upon consideration thereof.

### EXAMPLE 1

### Determination of Threshold Amount

In this example, methods of the invention were correlated with the clinical outcome in 49 patients who were previously diagnosed (using colonoscopy) as having colorectal cancer, and 100 patients who were diagnosed as not having colorectal cancer. The threshold amount (the amount at which patients below such amount can be identified as being relatively disease-free) for use in methods of the invention was empirically determined as described below.

Stool specimens were collected from patients and frozen. Each frozen stool specimen, weighing approximately 32 grams, was thawed and homogenized in buffer. The buffer was comprised of 0.5 M Tris, 10 mM NaCl, and 150 mM EDTA, essentially as disclosed in U.S. Pat. No. 6,551,777, incorporated by reference herein. Each of the samples was then diluted with additional buffer (not containing EDTA) to a final buffer to stool ratio of 20:1. Each sample was centrifuged, and the supernatant, which carried the active DNA degrading fraction, was removed to a clean tube. The supernatant was collected and treated with sodium dodecyl sulfate and Proteinase K. The DNA in each sample was then prepared by standard techniques. See, e.g., Ausubel et al., Short Protocols in Molecular Biology §§ 2.1-2.4 (3d ed. 1995). A phenol extraction, a phenol/chloroform extraction, and a phenol extraction were performed prior to isolating the DNA. The isolated DNA was then placed into a standard. Tris buffer.

The DNA samples were amplified using quantitative PCR. A PCR primer set from Midland Certified Reagent Company, TaqMan® probes from PanVera Corporation and a real-time PCR instrument were used (Biorad Corporations's iCycler iQ Real Time PCR Detection System). TaqMan® analysis was performed on an ABI 7700 thermalcycler (Applied Biosystems, Foster City, Calif.) using primers against a 200 base pair region of the APC gene. The 5′ primer was: 5′AGCCCCAGTGATCTTCCAGAT3′ (SEQ ID NO: 1). The ′3 primer was: 5′AGGTGGTGGAGGTGTTTTACTTCT3′ (SEQ ID NO: 2). A FAM/TAMRA probe was used to detect amplified PCR product: FAM-CCCTGGACAAACCATGCCACCAA-TAMRA (SEQ ID NO: 3).

Amplification reactions consisted of captured human stool DNA mixed with TaqMan® PCR Universal Master mix (Applied Biosystems, Foster City, Calif.), 1×PCR primers (5 μmol/L), and 1×TaqMan® probe (2 μmol/L) (Applied Biosystems, Foster City, Calif.). 5 mls of captured DNA were used in PCR reactions. TaqMan® reactions were performed as follows. Thermal cycling began with a primer annealing step (50° C. for 2 min) and one cycle of DNA denaturation (95° C. for 10 minutes). This was followed by 40 cycles of sequential DNA denaturation (95° C. for 1 min) and primer annealing (60° C. for 1 min). The ABI 7700 unit detected amplification products with the FAM/TAMRA probe and data used in the calculation of genome equivalents per reaction was provided. Clinical status was determined by performing a colonoscopy on each patient. The results are shown in the Table 1 below.

TABLE 1

| Patient No. | Clinical Status | GE |
|---|---|---|
| PV-11 | Normal | 147 |
| PV-64 | Normal | 155 |
| PV-109 | Normal | 163 |
| PV-10 | Normal | 168 |
| PV-27 | Normal | 180 |
| PV-56 | Normal | 266 |
| PV-119 | Normal | 272 |
| PV-59 | Normal | 312 |
| PV-137 | Normal | 334 |
| PV-8 | Minor Polyps | 386 |
| PV-52 | Normal | 394 |
| PV-96 | Normal | 404 |
| PV-44 | Normal | 490 |
| PV-3 | Minor Polyps | 498 |
| PV-57 | Minor polyps | 536 |
| PV-23 | Normal | 574 |
| PV-81 | Minor Polyps | 630 |
| PV-111 | Dukes A | 652 |
| PV-141 | Normal | 688 |
| PV-84 | Normal | 736 |
| PV-5 | Normal | 746 |
| PV-106 | Normal | 756 |
| PV-19 | Dukes A | 772 |
| PV-89 | Normal | 788 |
| PV-39 | Dukes C | 834 |
| PV-99 | Minor Polyps | 844 |
| PV-146 | Minor Polyps | 850 |
| PV-105 | Minor Polyps | 886 |
| PV-1 | Minor Polyps | 940 |
| PV-73 | Normal | 952 |
| PV-140 | Normal | 1,038 |
| PV-28 | Dukes B | 1,138 |
| PV-16 | Normal | 1,226 |
| PV-9 | Normal | 1,246 |
| PV-68 | Dukes A | 1,262 |
| PV-60 | Dukes B | 1,290 |
| PV-130 | Minor Polyps | 1,302 |
| PV-22 | Minor Polyps | 1,312 |
| PV-63 | Normal | 1,334 |
| PV-123 | Minor Polyps | 1,334 |
| PV-118 | Minor Polyps | 1,354 |
| PV-124 | Normal | 1,468 |
| PV-35 | Minor Polyps | 1,510 |
| PV-133 | Normal | 1,510 |
| PV-4 | Minor Polyps | 1,564 |
| PV-72 | Normal | 1,582 |
| PV-41 | Normal | 1,604 |
| PV-53 | Dukes A | 1,670 |
| PV-147 | Minor Polyps | 1,688 |
| PV-40 | Dukes C | 1,870 |
| PV-114 | Minor Polyps | 1,936 |
| PV-92 | Minor Polyps | 1,956 |
| PV-113 | Minor Polyps | 1,982 |
| PV-121 | Normal | 2,040 |
| PV-136 | Minor Polyps | 2,080 |
| PV-125 | Minor Polyps | 2,120 |
| PV-132 | Normal | 2,120 |
| PV-138 | Normal | 2,140 |
| PV-112 | Minor Polyps | 2,180 |
| PV-12 | Dukes A | 2,200 |
| PV-128 | Minor Polyps | 2,240 |
| PV-148 | Minor Polyps | 2,240 |
| PV-150 | Normal | 2,260 |
| PV-62 | Normal | 2,360 |
| PV-43 | Minor Polyps | 2,420 |
| PV-100 | Minor Polyps | 2,540 |
| PV-104 | Minor Polyps | 2,560 |
| PV-2 | Minor Polyps | 2,580 |
| PV-90 | Minor Polyps | 2,600 |
| PV-117 | LCD | 2,620 |
| PV-95 | Minor Polyps | 2,640 |
| PV-85 | Normal | 2,660 |
| PV-26 | Normal | 2,720 |
| PV-17 | Normal | 2,760 |
| PV-108 | Minor Polyps | 2,800 |
| PV-144 | Normal | 2,820 |
| PV-120 | Minor Polyps | 2,880 |
| PV-143 | Minor Polyps | 2,880 |
| PV-37 | Minor Polyps | 2,940 |
| PV-107 | Minor Polyps | 3,020 |
| PV-71 | Dukes B | 3,140 |
| PV-38 | Dukes C | 3,200 |
| PV-30 | Normal | 3,220 |
| PV-7 | Dukes A | 3,220 |
| PV-66 | Normal | 3,440 |
| PV-86 | Dukes C | 3,520 |
| PV-126 | Normal | 3,540 |
| PV-115 | Minor Polyps | 3,560 |
| PV-51 | Normal | 3 640 |
| PV-145 | Normal | 3,660 |
| PV-29 | Dukes B | 3,840 |
| PV-65 | Dukes B | 3,940 |
| PV-14 | Minor Polyps | 4,180 |
| PV-46 | Dukes C | 4,200 |
| PV-36 | Dukes A | 4,300 |
| PV-49 | Minor Polyps | 4,420 |
| PV-47 | Dukes C | 4,420 |
| PV-34 | Dukes A | 4,440 |
| PV-139 | Minor Polyps | 4,740 |
| PV-82 | Normal | 4,980 |
| PV-79 | Minor Polyps | 5,000 |
| PV-58 | Dukes B | 5,000 |
| PV-98 | Dukes A | 5,140 |
| PV-67 | Dukes A | 5,220 |
| PV-134 | Normal | 5,240 |
| PV-33 | Dukes D | 5,240 |
| PV-31 | CIS/HGD | 5,500 |
| PV-122 | Normal | 5,560 |
| PV-129 | Normal | 5,560 |
| PV-45 | Minor Polyps | 5,620 |
| PV-97 | Minor Polyps | 5,940 |
| PV-149 | Normal | 6,080 |
| PV-116 | Normal | 6,120 |
| PV-74 | Normal | 6,180 |
| PV-102 | Dukes A | 6,980 |
| PV-83 | Normal | 7,160 |
| PV-93 | Dukes C | 7,860 |
| PV-131 | Minor Polyps | 7,900 |
| PV-61 | Dukes B | 7,940 |
| PV-127 | Minor Polyps | 8,020 |
| PV-55 | Dukes C | 8,240 |
| PV-69 | Dukes B | 8,280 |
| PV-76 | Dukes D | 9,460 |
| PV-25 | Minor Polyps | 9,580 |
| PV-94 | Minor Polyps | 9,780 |
| PV-15 | Dukes A | 10,500 |
| PV-135 | Minor Polyps | 10,580 |
| PV-110 | Minor Polyps | 11,620 |
| PV-20 | Dukes B | 11,960 |
| PV-142 | Normal | 13,240 |
| PV-54 | Dukes C | 14,620 |
| PV-70 | Dukes C | 15,620 |
| PV-21 | Dukes B | 17,940 |
| PV-87 | Dukes C | 18,180 |
| PV-32 | Dukes C | 20,400 |
| PV-91 | Normal | 22,400 |
| PV-78 | Normal | 23,000 |
| PV-6 | Dukes A | 25,400 |
| PV-77 | Dukes C | 25,600 |
| PV-42 | Dukes C | 27,600 |
| PV-50 | Normal | 28,000 |
| PV-24 | Dukes B | 29,200 |
| PV-88 | Dukes C | 32,800 |
| PV-80 | Dukes A | 54,600 |
| PV-101 | Dukes B | 59,600 |
| PV-75 | Dukes C | 90,400 |
| PV-13 | Dukes A | 138,600 |
| PV-103 | Dukes C | 141,800 |
| PV-18 | Dukes A | 238,000 |
| PV-48 | CIS | 286,000 |

In reference to the table above, the diagnoses of “normal” or “minor polyps” are considered “normal patients” as discussed herein. Also, the diagnoses of “Dukes A,” “Dukes B,” “Dukes C” (refers to the stages of colorectal cancer), “LGD” (Low Grade Dysplasia), “HGD” (High Grade Dysplasia), and “CIS” (Carcinoma In Situ) are considered “cancer patients” as discussed herein.

As shown in the table above, there is overlap of GE scores for normal patients and cancer patients. The lowest GE score for a cancer patient was 652 and the lowest GE score for a normal patient was 147. However, there were 17 normal patients with GE scores lower than the lowest GE score for a cancer patient (652). Accordingly, using this data set, the predetermined threshold amount for use in methods of the invention can be set to any score below 652. As one example, the GE score can be set at 650. Based on this score, 17% of normal patients would not have to undergo additional disease testing. As another example, the GE score can be set at 630 (i.e., the highest number of GEs for a normal patient that is less than the lowest number of GEs for a cancer patient). Based on this score, 16% of normal patients would not have to undergo additional disease testing. As further example, the GE score can be set at 500 to eliminate potential false-negatives in future testing. Based on this score, 14% of normal patients would not have to undergo additional disease testing. As another example, the GE score can be set at 250. Based on this score, 9% of normal patients would not have to undergo additional disease testing. As a further example, the GE score can be set at 200. Based on this score, 5% of normal patients would not have to undergo additional disease testing. As a further example, the GE score can be set at 700 to eliminate potential false-positives in future testing.

Using methods of the invention, a subset of the patient population would not have to undergo additional disease testing based on GE scores below the predetermined threshold amount. To reduce the likelihood of false-negative results using methods of the invention, the same preparation methods, amplification methods, and quantitative analysis methods that were used to determined the threshold amount should be used when screening patient samples in accordance with the invention.

### EXAMPLE 2

According to methods of the invention, if the amount of DNA in a patient sample is greater than the predetermined threshold amount, then the patient is identified as a candidate for additional disease testing. For example, if the predetermined threshold amount is 1,000 GEs, and a patient sample measures 50,000 GEs, then the patient would be a candidate for additional disease testing. Example 3 describes another preferred method of additional disease testing in accordance with the invention.

### Additional Disease Testing: DNA Integrity Assay

Detection of DNA fragments of at least 200 base pairs in length are also useful in an additional disease testing phase of the invention, as the amount of 200 bp or greater DNA in a sample is predictive of cancer or precancer in patients. The samples are screened by hybrid capturing human DNA and determining the amount of amplifiable DNA having at least 200 base pairs. Stool samples are prepared as described in Example 1.

Human DNA is isolated from stool precipitate by sequence-specific hybrid capture. Biotynilated probes against portions of the p53, K-ras, and ape genes are used. The K-ras probe is 5′GTGGAGTATTTGATAGTGTATTAACCTTATGTGTGAC 3′ (SEQ ID NO: 4). There are two apc probes. The apc-1309 probe is 5′TTCCAGCAGTGTCACAGCACCCTAGAACCAAATCCAG 3′ (SEQ ID NO: 5), and the apc-1378 probe is 5′CAGATAGCCCTGGACAAACAATGCCACGAAGCAGAAG 3′ (SEQ ID NO: 6). There are four probes against p53. The first (hybridizing to a portion of exon 5) is 5′TACTCCCCTGCCCTCAACAAGATGTTTTGCCAACTGG3′ (SEQ ID NO: 7), the second (hybridizing to a portion of exon 7) is 5′ATTTCTTCCATACTACTACCCATCGACCTCTCATC3′ (SEQ ID NO: 8), the third, also hybridizing to a portion of exon 7 is 5′ATGAGGCCAGTGCGCCTTGGGGAGACCTGTGGCAAGC3′ (SEQ ID NO: 9); and finally, a probe against exon 8 has the sequence 5′GAAAGGACAAGGGTGGTTGGGAGTAGATGGAGCCTGG3′ (SEQ ID NO: 10). A 10 μl aliquot of each probe (20 pmol/capture) is added to a suspension containing 300 μl DNA in the presence of 310 μl 6M GITC buffer for 2 hours at room temperature. Hybrid complexes are isolated using streptavidin-coated beads (Dynal). After washing, probe-bead complexes are suspended at 25° C. for 1 hour in 0.1×TE buffer, pH7.4. The suspension is then heated for 4 minutes at 85° C., and the beads are removed.

Each sample is amplified using forward and reverse primers through 7 loci (Kras, exon 1, APC exon 15 (3 separate loci), p53, exon 5, p53, exon 7, and p53, exon 8) in duplicate (for a total of 14 amplifications for each locus). Seven separate PCRs (33 cycles each) are run in duplicate using primers directed to detect fragments in the sample having 200 base pairs or more. Amplified DNA is placed on a 4% Nusieve (FMC Biochemical) gel (3% Nusieve, 1% agarose), and stained with ethidium bromide (0.5 μg/ml). The resulting amplified DNA is graded based upon the relative intensity of the stained gels. Samples from a patient with cancer or adenoma are detected as a band having significantly greater intensity than the bands associated with samples from patients who do not have cancer or precancer. Patients are identified as having cancer or adenoma by determining the amount of amplifiable DNA measuring 200 base pairs or greater in length.

### EXAMPLE 3

According to methods of the invention, if the amount of DNA in a patient sample is greater than the predetermined threshold amount, then the patient is identified as a candidate for additional disease testing. For example, if the predetermined threshold amount is 500 GEs, and a patient sample measures 20,000 GEs, then the patient would be a candidate for additional disease testing. Example 2 describes a preferred method of additional disease testing in accordance with the invention.

### Additional Disease Testing: Detection of BAT-26 Mutation

The BAT-26 segment of the MSH1 mismatch repair locus (shown in SEQ ID NO: 11) is useful in the additional disease testing phase of the invention, as-deletions in BAT-26 have been associated with colorectal cancer. Stool samples are prepared as described in Example 1.

A primer is hybridized to the portion of the BAT-26 locus immediately upstream of the poly-A tract, which consists of 26 adenosines (nucleotides 195-221). Unlabeled deoxythymidine, a mixture of labeled and unlabeled deoxycytosine, and unlabeled dideoxyadenine are added along with polymerase. The primer is extended through the poly-A region. The labeled and unlabelled cytosine is extended for the next three bases (nucleotides 222-224, all guanines in the intact sequence) such that label is incorporated into each extended primer. After the poly-A tract and the three guanines, there exist two thymidines in the intact sequence. Thus, the dideoxyadenosine stops primer extension by addition at the end of a primer that is extended through the poly-A and triguanine regions. Strands are separated, and the length of the strands are observed on a polyacrylamide gel to detect deletions in the poly-A tract. Deletions in the poly-A tract are indicative of colorectal cancer.

Although details of the present invention have been described with reference to specific and preferred embodiments, it should be understood by those skilled in the art that various changes in form and detail may be made therein without departing from the spirit and scope of the invention.

What is claimed is:

1. A method for screening a patient for the presence of disease, comprising the steps of:
measuring a quantitative amount of nucleic acid in a patient sample comprising shed cells or cellular debris; and
identifying the patient as a candidate for additional disease testing if the amount of nucleic acid is above a predetermined threshold amount.

2. The method of claim 1, wherein the nucleic acid is genomic DNA.

3. The method of claim 1, wherein the measuring comprises determining a number of genome equivalents.

4. The method of claim 1, further comprising the step of performing an assay on a sample from the patient if the patient is identified as a candidate for additional disease testing.

5. The method of claim 4, wherein the assay is selected from the group consisting of a DNA integrity assay, mutation detection, enumerated LOH, expression assays, and FISH.

6. The method of claim 4, wherein the assay detects mutations at a genetic locus selected from the group consisting of p53, ras, APC, DCC, and BAT-26.

7. The method of claim 1, further comprising the step of performing a diagnostic examination on the patient if the patient is identified as a candidate for additional disease testing.

8. The method of claim 7, wherein the step of performing a diagnostic examination is selected from a group consisting of a colonoscopy, a sigmoidoscopy, a fecal occult blood testing and an upper gastrointestinal evaluation.

9. The method of claim 1, wherein the patient sample is stool.

10. The method of claim 1, wherein the patient sample is selected from the group consisting of sputum, pancreatic fluid, bile, lymph, blood, urine, cerebrospinal fluid, seminal fluid, saliva, breast nipple aspirate, and pus.

11. The method of claim 1, wherein the disease is cancer or pre-cancer.

12. The method of claim 11, wherein the cancer is colorectal cancer.

13. The method of claim 11, wherein the cancer is selected from the group consisting of lung cancer, esophageal cancer, prostrate cancer, stomach cancer, pancreatic cancer, liver cancer, and lymphoma.

14. A method for screening a patient for the presence of abnormal proliferating cells, comprising the steps of:
measuring a quantitative amount of nucleic acid in a patient sample comprising shed cells or cellular debris; and
identifying a positive screen as a sample in which the amount of nucleic acid is above a predetermined threshold amount.

15. The method of claim 14, wherein the nucleic acid is genomic DNA.

16. The method of claim 14, wherein the measuring comprises determining a number of genome equivalents.

17. The method of claim 14, further comprising the step of performing an assay on a sample from the patient if a positive screen is identified in the identifying step.

18. The method of claim 17, wherein the assay is selected from the group consisting of a DNA integrity assay, mutation detection, enumerated LOH, expression assays, and FISH.

19. The method of claim 17, wherein the assay detects mutations at a genetic locus selected from the group consisting of p53, ras, APC, DCC, and BAT-26.

20. The method of claim 14, further comprising the step of performing a diagnostic examination on the patient if a positive screen is identified in the identifying step.

21. The method of claim 20, wherein the step of performing a diagnostic examination is selected from a group consisting of a colonoscopy, a sigmoidoscopy, a fecal occult blood testing and an upper gastrointestinal evaluation.

22. The method of claim 14, wherein the patient sample is stool.

23. The method of claim 14, wherein the patient sample is selected from the group consisting of sputum, pancreatic fluid, bile, lymph, blood, urine, cerebrospinal fluid, seminal fluid, saliva, breast nipple aspirate, and pus.

24. A method for diagnosing the state of health of a patient, comprising the steps of:
measuring a quantitative amount of nucleic acid in a patient sample comprising shed cells or cellular debris; and
performing an assay on a sample from the patient if the amount of nucleic acid is above a predetermined threshold amount, wherein the state of health of a patient is determined.

25. The method of claim 24, wherein the nucleic acid is genomic DNA.

26. The method of claim 24, wherein the measuring comprises determining a number of genome equivalents.

27. The method of claim 24, wherein the assay is selected from the group consisting of a DNA integrity assay, mutation detection, enumerated LOH, expression assays, and FISH.

28. The method of claim 24, wherein the assay detects mutations at a genetic locus selected from the group consisting of p53, ras, APC, DCC, and BAT-26.

29. The method of claim 24, wherein the method further comprises performing a diagnostic examination on the patient.

30. The method of claim 29, wherein the diagnostic examination is selected from a group consisting of a colonoscopy, a sigmoidoscopy, a fecal occult blood testing and an upper gastrointestinal evaluation.

31. The method of claim 24, wherein the patient sample is stool.

32. The method of claim 24, wherein the patient sample is selected from the group consisting of sputum, pancreatic fluid, bile, lymph, blood, urine, cerebrospinal fluid, seminal fluid, saliva, breast nipple aspirate, and pus.
